(19) United States
(12) Patent Application Publication
(10) Pub. No.: US 2005/0209248 A1
(43) Pub. Date: Sep. 22, 2005

(54) Plymorphic forms of phenyl oxazolidinone derivatives

(76) Inventors: Biswajit Das, Haryana (IN); Anita Mehta, Buffalo Grove, IL (US); Ashok Rattan, Delhi (IN)

Correspondence Address: RANBAXY INC., 600 COLLEGE ROAD EAST, SUITE 2100, PRINCETON, NJ, 08540, US

(21) Appl. No.: 10/514,074

(22) PCT Filed: May 15, 2003
(86) PCT No.: PCT/IB03/01853

§ 371 Date: May 20, 2005

(30) Foreign Application Priority Data
May 15, 2002 (US) 60380606

(51) Int. Cl. A61K 31/496; C07D 413/14

(52) U.S. Cl. 514/254.02; 544/369

(57) ABSTRACT

The invention relates to phenyl oxazolidinone derivatives. More particularly, it relates to polymorphic forms of (S)-N-[[3-fluoro-4-[N-1[4-{2-furyl-(5-nitro)methyl}]piperazinyl]-phenyl]-2-oxo-5-oxazolidinyl]-methyl]acetamide hydrochloride having the Formula I. Further, the invention relates to methods of using such compounds as antimicrobials, pharmaceutical compositions containing the novel polymorphic forms, and processes for the preparation of the polymorphic forms.

### FIELD OF INVENTION

The invention relates to phenyl oxazolidinone derivatives. More particularly, it relates to polymorphic forms of (S)-N-[[3-fluoro-4-[N-1[4-{2-furyl-(5-nitro)methyl}]piperazinyl]-phenyl]-2-oxo-5-oxazolidinyl]-methyl]acetamide hydrochloride having the Formula I. 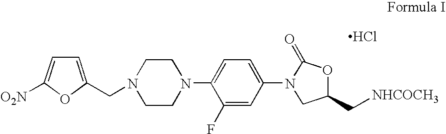
Further, the invention relates to methods of using such compounds as antimicrobials, pharmaceutical compositions containing the novel polymorphic forms, and processes for the preparation of the polymorphic forms.

### BACKGROUND OF THE INVENTION

S. epidermidis is the causative agent in many incidents of infection of implanted medical devices such as catheters, pacemakers, prosthetics joints, cardiac valves and central venous system shunts. These infections often recur and tend to be difficult to treat with antibiotics agents. Removal of the devices with concurrent administration of antibiotics is usually the only method of eradicating the focus of infection.

The compound of Formula I, namely, (S)-N-[[3-fluoro-4-[N-1 [4-{2-furyl-(5-nitro)methyl}] piperazinyl]-phenyl]-2-oxo-5-oxazolidinyl]-methyl]acetamide hydrochloride is a phenyl oxazolidinone derivative, as disclosed in PCT application WO 02/06278. It is said to be useful as antimicrobial agent, effective against a number of human and veterinary pathogens, including gram-positive aerobic bacteria, such as multiply resistant staphylococci, streptococci and enterococci as well as anaerobic organisms such as Bacterioides spp. and Clostridia spp. species, and acid fast organisms such as Mycobacterium tuberculosis, Mycobacterium avium and Mycobacterium spp.

The PCT application WO 02/06278 describes the preparation of compounds of Formula I. The products of Formula I obtained by following the cited methods tend to be hygroscopic and difficult to filter. These types of disadvantageous properties have proven to be serious obstacles to the large-scale manufacture of a compound. Further, handling problems are encountered during the preparation of pharmaceutical compositions comprising the hygroscopic compound of Formula I obtained by following the method disclosed in WO 02/06278.

### SUMMARY OF THE INVENTION

Provided herein is means to prepare a compound of Formula I in a form, which is non-hygroscopic, permits large scale, synthesis and which can overcome the handling problems encountered during the preparation of pharmaceutical compositions. There is a need to discover and develop a new agent active against all anaerobes including drug resistant strains.

Herein are provided new polymorphic forms of S)-N-[[3-fluoro-4-[N-1 [4-{2-furyl-(5-nitro)methyl}]piperazinyl]-phenyl]-2-oxo-5-oxazolidinyl]-methyl]acetamide hydrochloride (Formula I) designated as ‘Form A’and ‘Form B.’ Processes for the preparation of new polymorphic forms are also provided. Additionally, pharmaceutical formulations comprising polymorphic forms A and/or B and methods of using them as antimicrobial agents, agents for treating or preventing anaerobic infections, catheter infections and foreign body or prosthesis infections in mammals are provided. Further, ‘Form A’ is very active against slime-producing bacteria and retains activity against adherent bacteria, making it useful for the prevention and treatment of catheter infections and foreign body or prosthesis infections.

The polymorphic forms of the compound of Formula I designated as ‘Form A’ and ‘Form B’ can be characterized by their X-ray powder diffraction patterns (XRPD), infrared spectra and differential scanning calorimetry (DSC) characteristics.

Accordingly, polymorphic ‘Form A’ of the compound of Formula I and a process for the preparation of polymorphic ‘Form A’ are provided. This process comprises:
(i) providing free base of Formula I,
(ii) dissolving the free base of Formula I in ethanol,
(iii) adding ethanolic HCl (ethanol containing from about 2-10N hydrochloric acid) at about 40-55° C.,
(iv) cooling the resulting solution slowly to below room temperature, for example, about 10° C. and stirring at this temperature over a period of 4-6 hour,
(v) filtering the separated solid and digesting the solid in ethanol at 70-80° C. for 4-6 hours, and
(vi) cooling to below room temperature, for example about 10° C., filtering and drying the product under vacuum at about 50-75° C. to produce ‘Form A’ which can be characterized, for example, by the following data:
   Infrared absorption bands (cm⁻¹): 3421, 3286, 2967, 1747, 1722, 1668, 1524, 1504, 1416, 1354, 1327, 1272, 1242, 1170, 1106, 1078, 1022, 811, 749 (FIG. 1).
   X-ray powder diffraction (2θ): 6.58, 11.34, 12.86, 13.20, 13.40, 14.06, 14.32, 14.74, 15.26, 15.46, 15.91, 16.22, 16.46, 16.84, 17.22, 17.62, 18.16, 18.38, 18.84, 19.14, 19.74, 20.00, 20.60, 20.90, 21.18, 21.94, 22.48, 22.84, 23.52, 23.86, 24.08, 24.72, 25.08, 25.56, 25.90, 26.20, 26.62, 27.04, 27.80, 28.14, 28.48, 28.68, 29.12, 29.70, 30.10, 30.88, 31.48, 32.40, 33.50, 34.24 (FIG. 2).
   DSC: Endotherm at 211.93° C. (onset at 206.58° C.) (FIG. 3)

In another aspect, there is provided a polymorphic ‘Form B’ of the compound of Formula I and a process for the preparation of polymorphic ‘Form B’. This process comprises:
(i) providing free base of Formula I,
(ii) dissolving the free base of Formula I in hot ethanol (for example, ethanol at temperatures from about 60-80° C.),
(iii) cooling the solution to room temperature or below, for example, about 20° C.,
(iv) adding the ethanolic HCl (ethanol containing about 2-10N hydrochloric acid) at this temperature,
(v) stirring the reaction mixture at this temperature for about 15 minutes, and
(vi) filtering the separated solid to produce ‘Form B’ which can be characterized, for example, by the following data:
   Infrared absorption bands (cm⁻¹): 3423.2, 2386, 1747, 1654.3, 1519, 1425.9, 1356.2, 1239.2, 1022, 972.1, 811.7, 750.2 (FIG. 4).
   X-ray powder diffraction (2θ); 15.9, 19.12, 19.44, 20.22, 23.14, 25.66, 26.52, 28.46 (FIG. 5).
   DSC: Endotherms at 154.92° C. (onset at 148.26° C.) and at 209.22° C. (onset at 207.51° C.) (FIG. 6).

According to another embodiment, there is provided a process for the preparation of polymorphic ‘Form A’ of the compound of Formula I, which comprises:
(i) providing free base of Formula I,
(ii) dissolving free base of Formula I in ethanol while heating to about 60-80° C.,
(iii) adding a mixture of HCl in ethanol (about 2-10N), below room temperature, for example, at about 5° C.,
(iv) stirring the reaction mixture at about 5-15° C. for about 1-3 hours,
(v) removing the solvent and digesting the residue in dichloromethane,
(vi) filtering and crystallizing the solid from methanol/isopropyl alcohol mixtures, for example, in a range of about 4:1 to about 20:1,
(vii) digesting the solid in ethanol at about 60-80° C. for about 4 hours, and
(viii) cooling it to about 25-30° C., filtering and drying under vacuum at about 50-75° C. to produce ‘Form A’ which can be characterized by the data presented earlier for ‘Form A’.

According to another embodiment, there is provided a process for the preparation of novel polymorphic ‘Form A’ of the compound of Formula I, which comprises:
(i) dissolving compound of Formula I in de-mineralized water while heating to about 40-60° C.,
(ii) cooling the solution slightly to about 35-45° C.,
(iii) adding isopropyl alcohol at 25-30° C.,
(iv) stirring, filtering and washing the solid with isopropyl alcohol,
(v) drying under vacuum at about 60° C. to produce ‘Form A’ which can be characterized by the data presented earlier for ‘Form A’.

According to another embodiment, there is provided a process for the preparation of novel polymorphic ‘Form A’ of the compound of Formula I, which comprises:
(i) dissolving compound of Formula I in de-mineralized water while heating to about 40-60° C.,
(ii) cooling the solution slightly to about room temperature or slightly above,
(iii) adding ethanol at room temperature or slightly above, for example, about 25-30° C.,
(iv) stirring, cooling the reaction mixture to 10-15° C., filtering and washing the solid with ethanol, and
(v) drying under vacuum at about 60° C. to produce ‘Form A’ which can be characterized by the data presented earlier for ‘Form A’.

### BRIEF DESCRIPTION OF THE DRAWINGS

Embodiments of the invention are explained in greater detail by way of the accompanying figures:

FIG. 1 is an infrared spectrum (IR) showing a spectrum of ‘Form A’ of compound of Formula I taken from the compound prepared according to Example 1.

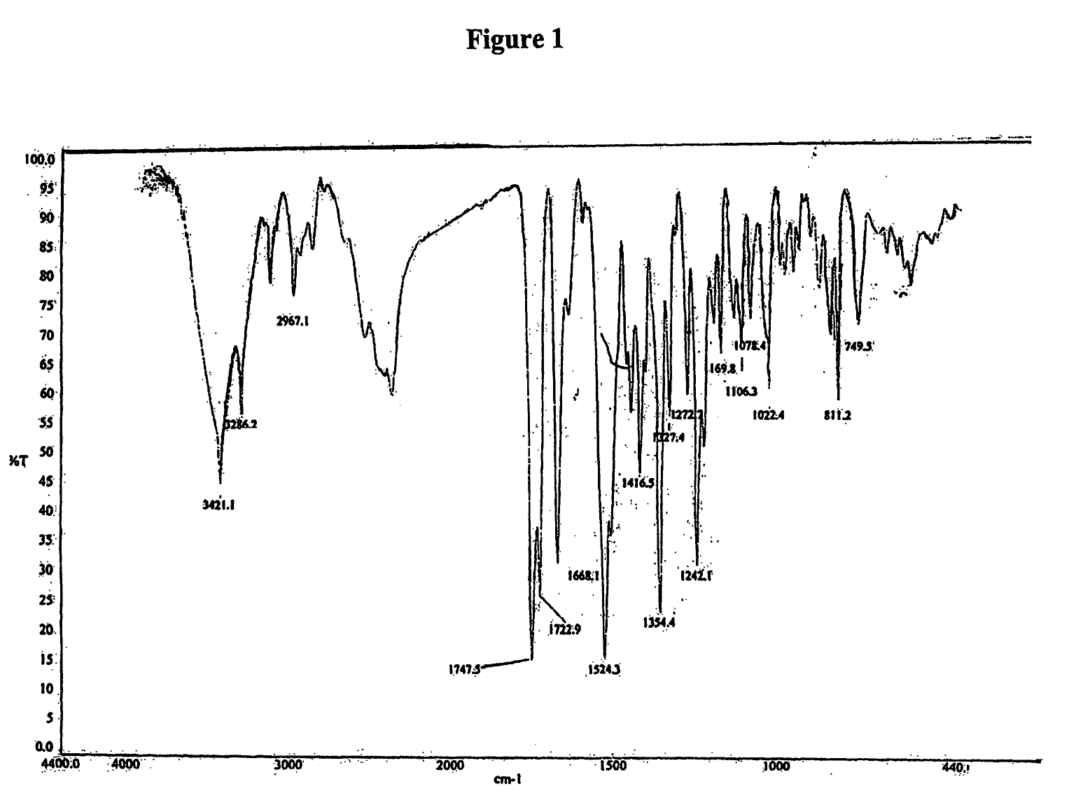

FIG. 2 is a powder X-ray diffraction pattern (XRPD) of ‘Form A’ of compound of Formula I taken from the compound prepared according to Example 1.

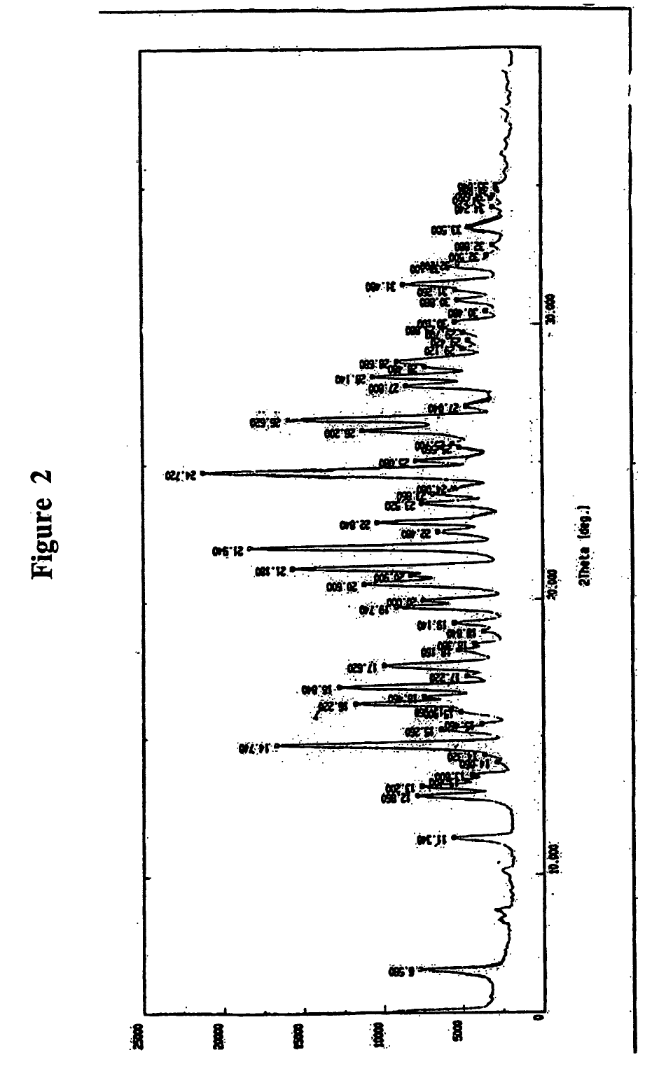

FIG. 3 is a differential scanning calorimetric (DSC) thermogram of ‘Form A’ of Formula I taken from the compound prepared according to Example 1.

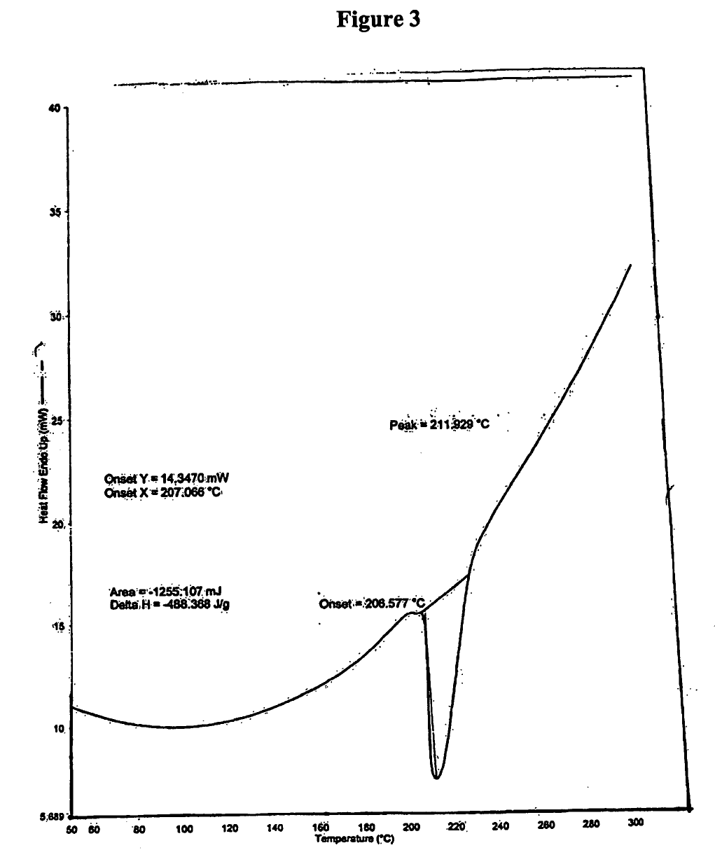

FIG. 4 is an infrared spectrum (IR) showing a spectrum of ‘Form B’ of compound of Formula I taken from the compound prepared according to Example 2.

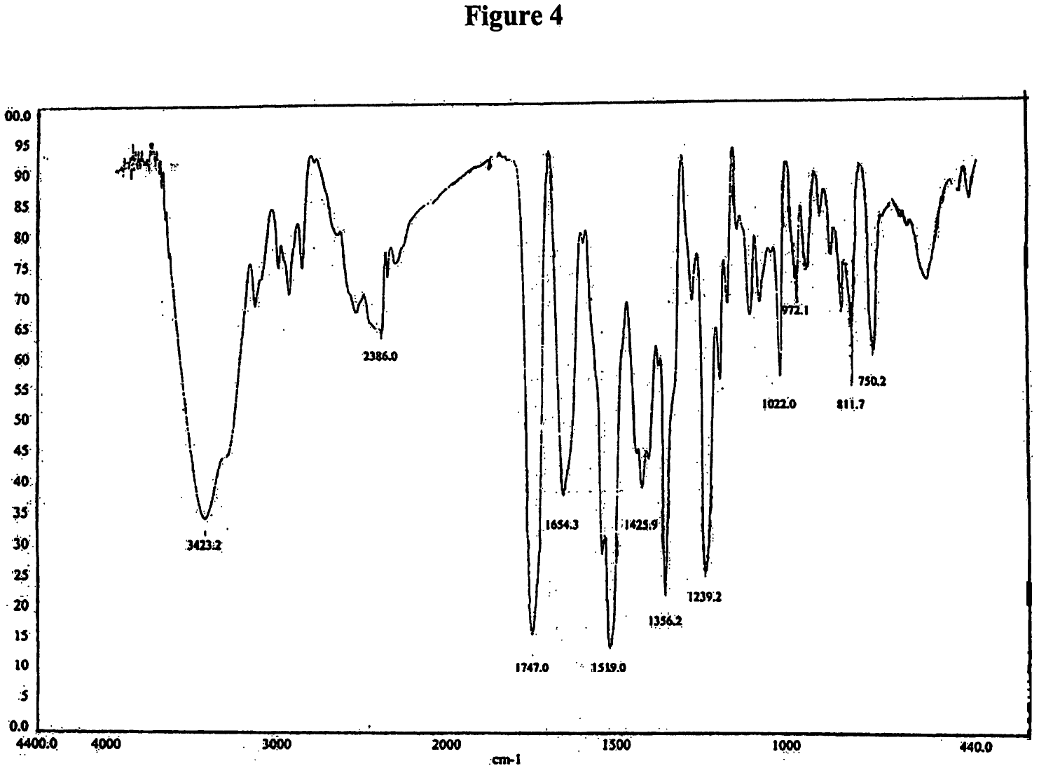

FIG. 5 is a powder X-ray diffraction pattern (XRPD) of ‘Form B’ of compound of Formula I taken from the compound prepared according to Example 2.

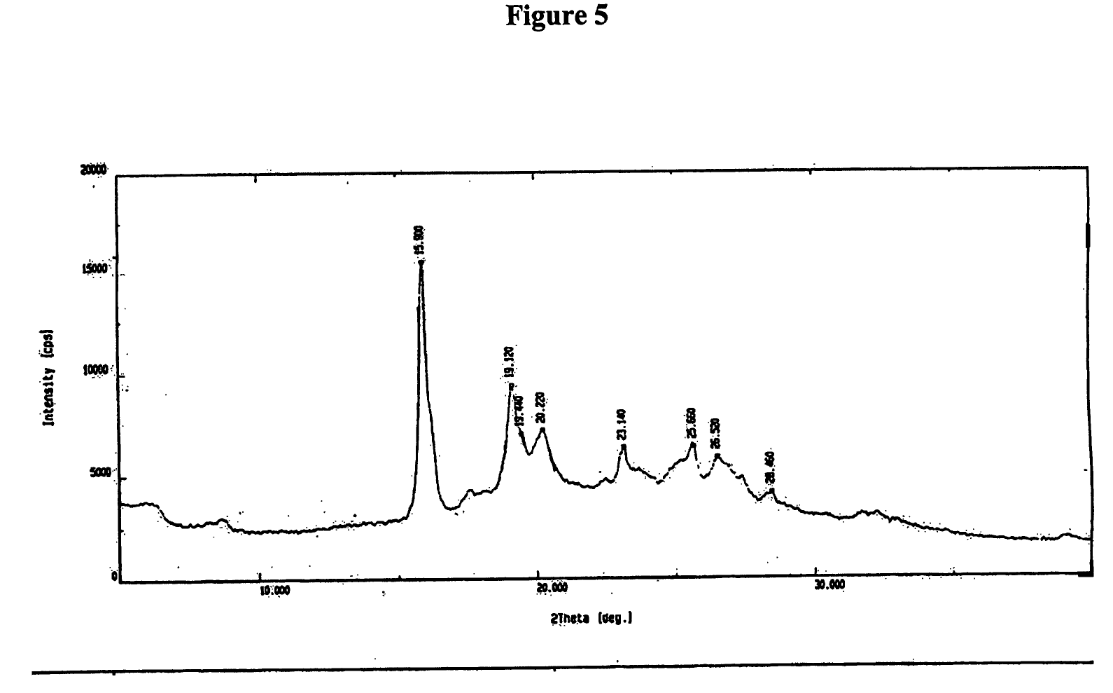

FIG. 6 is a differential scanning calorimetric (DSC) thermogram of ‘Form B’ of compound of Formula I taken from the compound prepared according to Example 2.

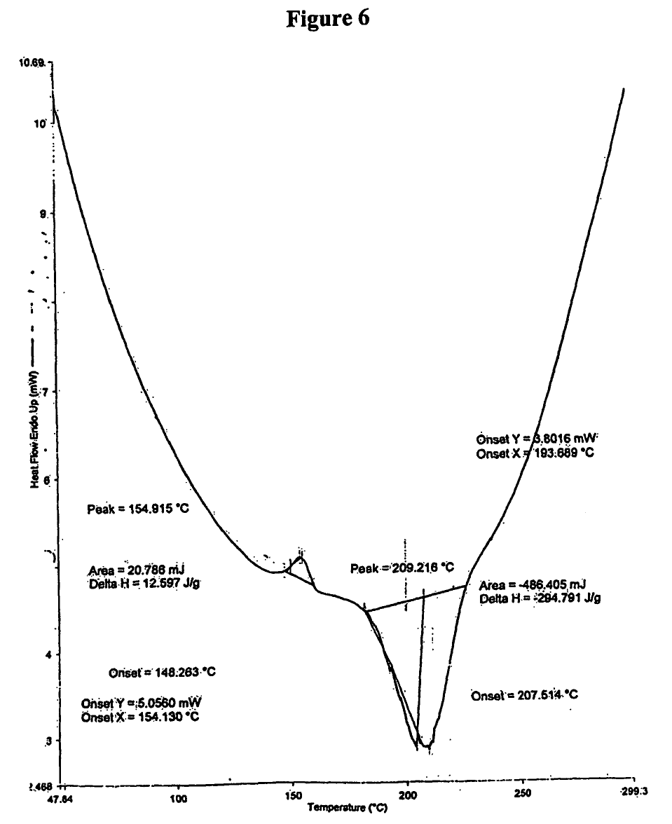

### DETAILED DESCRIPTION OF THE INVENTION

Data were collected as follows:

XRD: Instrument: Model RU-H3R (Rigaku)
Data collection parameters: Voltage: 50 KV; Current: 120 mA; Scan speed: 2°/min; Scan step: 0.02°; Scan range: 3-40°. XRD data on a compound prepared according to Example 1 is presented in Table I. Asterisks show the 20 most intense XRD peaks.
IR: Instrument: FTIR Paragon 1000PC
Data collection parameters: Medium: KBr; Scanning range: 440-4400 cm⁻¹.
DSC: Instrument: Perkin Elmer Pyris 1

Data collection parameters: Scanning rate: 10° C./min; Temperature: 50° C.-300° C.

TABLE I

| | X-ray powder diffraction |
|---|---|
| S. No. | (2θ) |
| 1. | 6.580 |
| 2. | 11.340 |
| 3. | 12.860* |
| 4. | 13.200* |
| 5. | 13.400 |
| 6. | 14.060 |
| 7. | 14.320 |
| 8. | 14.740* |
| 9. | 15.260 |
| 10. | 15.460 |
| 11. | 15.909 |
| 12. | 16.220* |
| 13. | 16.460 |
| 14. | 16.840* |
| 15. | 17.220 |
| 16. | 17.620* |
| 17. | 18.160 |
| 18. | 18.380 |
| 19. | 18.840 |
| 20. | 19.140 |
| 21. | 19.740* |
| 22. | 20.000* |
| 23. | 20.600* |
| 24. | 20.900 |
| 25. | 21.180* |
| 26. | 21.940* |
| 27. | 22.480* |
| 28. | 22.840* |
| 29. | 23.520* |
| 30. | 23.860 |
| 31. | 24.080 |
| 32. | 24.720* |
| 33. | 25.080 |
| 34. | 25.560 |
| 35. | 25.900 |
| 36. | 26.200* |
| 37. | 26.620* |
| 38. | 27.040 |
| 39. | 27.800 |
| 40. | 28.140* |
| 41. | 28.480 |
| 42. | 28.680* |
| 43. | 29.120 |
| 44. | 29.700 |
| 45. | 30.100 |
| 46. | 30.880 |
| 47. | 31.480* |
| 48. | 32.400 |
| 49. | 33.500 |
| 50. | 34.240 |

Biological Activity
Activity Against Anaerobes and microbacterium
Agar dilution method for anaerobic bacteria:

MICs were determined by the NCCLS agar dilution method with Wilkins Chalgren Agar (Difco). The plates were incubated in an anaerobic jar containing an atmosphere of 85% nitrogen, 10% hydrogen and 5% carbon dioxide for 48 hour. MIC values are presented in Table II.

TABLE II

| Antibiotics | MIC₅₀ | MIC₉₀ | Geometric Mean | MIC Range |
|---|---|---|---|---|
| Polymorphic | 0.032 | 0.25 | 0.037 | 0.004-1 |
| ‘Form A’ | | | | |
| Linezolid | 1 | 4 | 1.134 | 0.25-4 |
| Vancomycin | 32 | 32 | 9.306 | 0.5-32 |
| Teicoplanin | 2 | 32 | 2.04 | 0.03-32 |
| Synercid | 1 | 16 | 1.614 | 0.062-16 |
| Amox | 1 | 256 | 1.366 | 0.062-256 |
| Amox + clav | 0.25 | 8 | 0.423 | 0.062-32 |
| Imipenem | 0.064 | 1 | 0.084 | 0.008-4 |
| Clindamycin | 0.125 | 8 | 0.208 | 0.008-64 |
| Metronidazole | 0.5 | 2 | 0.48 | 0.062-32 |
| Gatifloxacin | 0.5 | 2 | 0.659 | 0.06-32 |
| Moxifloxacin | 0.5 | 2 | 0.566 | 0.03-32 |

Some of the MICs obtained are presented in Table III.

TABLE III

| | Polymorphic | | | | | |
|---|---|---|---|---|---|---|
| Organism | ‘Form A’ | Linezolid | Vanco | Teico | Quin/dal | Amox |
| Clostridium camis | 0.03 | 2 | 2 | <=.06 | 0.5 | <=.125 |
| Clostridium camis | 0.016 | 2 | 2 | <=.06 | 0.5 | <=.125 |
| Clostridium | 0.03 | 2 | 0.5 | <=.06 | 0.5 | <=.125 |
| perfringens | | | | | | |
| Clostridium | 0.03 | 2 | 0.5 | <=.06 | 0.5 | <=.125 |
| perfringens | | | | | | |
| Clostridium difficile | 0.03 | 2 | 2 | 0.25 | 0.5 | 1 |
| Clostridium difficile | 0.03 | 2 | 4 | 0.25 | 0.5 | 2 |
| Bacteroides fragilis | 0.03 | 4 | >16 | >16 | 8 | 32 |
| Bacteroides fragilis | 0.06 | 4 | >16 | >16 | >8 | >128 |
| Bacteroides fragilis | 0.06 | 4 | >16 | >16 | >8 | >128 |
| Preotella | 0.125 | 4 | >16 | 16 | >8 | >128 |
| (Bacteroides) | | | | | | |
| disiens | | | | | | |
| Prevotella | 0.06 | 4 | >16 | >16 | 8 | >128 |
| (Bacteroides) | | | | | | |
| disiens | | | | | | |
| Prevotella bivia | 0.125 | 1 | >16 | 1 | 2 | <=.125 |
| Prevotella | 0.016 | 0.5 | >16 | 0.5 | 0.25 | 4 |
| intermedia | | | | | | |
| Prevotella | 0.016 | 1 | >16 | 0.5 | 0.25 | <=.125 |
| intermedia | | | | | | |
| Prevotella | 0.06 | 1 | >16 | 2 | 1 | <=.125 |
| melaninogenica | | | | | | |
| Prevotella | 0.125 | 2 | >16 | 4 | 2 | 64 |
| melaninogenica | | | | | | |
| Porphyromonas | <=.008 | 1 | 2 | 0.125 | <=.125 | <=.125 |
| asaccharolytica | | | | | | |
| Fusobacterium | 0.03 | 0.25 | >16 | >16 | 8 | 128 |
| montiferum | | | | | | |
| Fusobacterium | 0.03 | 0.25 | >16 | >16 | >8 | >128 |
| montiferum | | | | | | |
| Fusobacterium | 0.03 | 0.25 | >16 | >16 | >8 | 1 |
| montiferum | | | | | | |
| Fusobacterium | 0.03 | 0.25 | >16 | >16 | 4 | 1 |
| montiferum | | | | | | |
| Fusobacterium | <=.008 | 0.5 | >16 | >16 | 2 | <=.125 |
| nucleatum | | | | | | |
| Fusobacterium | 0.016 | 0.5 | >16 | >16 | 1 | <=.125 |
| nucleatum | | | | | | |
| Fusobacterium | 0.016 | 0.5 | >16 | >16 | 1 | <=.125 |
| nucleatum | | | | | | |
| Fusobacterium | 0.016 | 1 | >16 | >16 | 4 | <=.125 |
| nucleatum | | | | | | |
| Porphyromonas | <=.008 | 1 | 8 | <=.06 | 0.25 | <=.125 |
| gingivalis | | | | | | |
| Fusobacterium | 1 | 1 | >16 | >16 | >8 | 1 |
| varium | | | | | | |
| Fusobacterium | 0.25 | 1 | >16 | >16 | >8 | 1 |
| varium | | | | | | |
| P acnes | 1 | 0.5 | 0.5 | 0.25 | <=.125 | <=.125 |
| P acnes | 1 | 0.5 | 1 | 0.25 | <=.125 | <=.125 |
| P acnes | 1 | 0.5 | 0.5 | 0.25 | <=.125 | <=.125 |
| P acnes | 1 | 0.5 | 0.5 | 0.25 | <=.125 | 0.25 |
| Peptostreptococcus | <=.008 | 0.5 | 0.5 | 0.125 | <=.125 | 0.25 |
| asaccharolyticus | | | | | | |
| Fusobacterium | 0.5 | 1 | >16 | >16 | >8 | 1 |
| varium | | | | | | |
| Peptostreptococcus | <=.008 | 1 | 0.125 | 0.125 | 0.25 | <=.125 |
| asaccharolyticus | | | | | | |
| Peptostreptococcus | 0.016 | 2 | 0.5 | 0.125 | 0.25 | 0.25 |
| magnum | | | | | | |
| Peptostreptococcus | <=.008 | 1 | 0.25 | <=.06 | 0.25 | <=.125 |
| magnum | | | | | | |
| Peptostreptococcus | 0.016 | 1 | 0.25 | 0.125 | 0.25 | 0.25 |
| magnum | | | | | | |
| Peptostreptococcus | <=.008 | 2 | 0.25 | 0.125 | 0.25 | 0.5 |
| magnum | | | | | | |
| Peptostreptococcus | <=.008 | 0.5 | 1 | 0.125 | 0.5 | <=.125 |
| micros | | | | | | |
| Peptostreptococcus | 0.016 | 1 | 1 | <=.06 | 1 | <=.125 |
| micros | | | | | | |
| Peptostreptococcus | 0.016 | 1 | 1 | <=.06 | 0.5 | <=.125 |
| micros | | | | | | |
| Peptostreptococcus | 0.016 | 0.5 | 1 | 0.125 | 1 | <=.125 |
| micros | | | | | | |
| Peptostreptococcus | <=.008 | 0.5 | 1 | 0.125 | 1 | <=.125 |
| tetradius | | | | | | |
| Peptostreptococcus | <=.008 | 0.5 | 1 | <=.06 | 1 | <=.125 |
| tetradius | | | | | | |
| Peptostreptococcus | 0.016 | 0.5 | 0.125 | 0.25 | 0.25 | <=.125 |
| prevotii | | | | | | |
| Peptostreptococcus | <=.008 | 0.5 | 0.125 | <=.06 | 0.25 | 0.25 |
| prevotii | | | | | | |
| Eubacterium leutum | <=.008 | 1 | 1 | <=.06 | 0.25 | 1 |
| Eubacterium leutum | <=.008 | 1 | 1 | 0.125 | 0.25 | 1 |
| Eubacterium leutum | <=.008 | 1 | 1 | 0.125 | 0.25 | 1 |
| Eubacterium leutum | <=.008 | 1 | 1 | 0.125 | 0.25 | 1 |
| Fusobacterium | <=.008 | 0.5 | >16 | >16 | 0.25 | 0.5 |
| necrogenes | | | | | | |

| Organism | Ax/clav | Imipen | Clinda | Metron | Gati | Moxi | Cefinase |
|---|---|---|---|---|---|---|---|
| Clostridium camis | <=.125 | 0.06 | 0.03 | <=.125 | 0.25 | 0.25 | − |
| Clostridium camis | <=.125 | 0.06 | 0.03 | <=.125 | 0.25 | 0.25 | − |
| Clostridium | <=.125 | 0.06 | 1 | 1 | 1 | 0.5 | − |
| perfringens | | | | | | | |
| Clostridium | <=.125 | 0.25 | 0.5 | 1 | 1 | 0.5 | − |
| perfringens | | | | | | | |
| Clostridium difficile | 1 | 4 | 2 | 0.25 | 1 | 1 | − |
| Clostridium difficile | 1 | 4 | 4 | 0.25 | 2 | 2 | − |
| Bacteroides fragilis | 0.5 | 0.06 | 0.5 | 0.5 | 1 | 0.25 | + |
| Bacteroides fragilis | 4 | 0.25 | 2 | 1 | 1 | 0.5 | + |
| Bacteroides fragilis | 8 | 0.5 | 1 | 1 | 1 | 0.5 | + |
| Preotella | 32 | 0.5 | 8 | 0.5 | 1 | 0.25 | + |
| (Bacteroides) | | | | | | | |
| disiens | | | | | | | |
| Prevotella | 8 | 0.03 | 4 | 1 | 1 | 0.5 | + |
| (Bacteroides) | | | | | | | |
| disiens | | | | | | | |
| Prevotella bivia | <=.125 | 0.03 | >32 | 1 | 2 | 2 | − |
| Prevotella | <=.125 | <=.016 | <=.016 | 0.5 | 0.25 | 0.5 | + |
| intermedia | | | | | | | |
| Prevotella | <=.125 | <=.016 | <=.016 | 0.25 | 0.25 | 0.5 | − |
| intermedia | | | | | | | |
| Prevotella | <=.125 | <=.016 | <=.016 | 0.25 | 0.5 | 1 | − |
| melaninogenica | | | | | | | |
| Prevotella | 2 | 0.03 | 0.03 | 0.5 | 8 | 16 | + |
| melaninogenica | | | | | | | |
| Porphyromonas | <=.125 | 0.03 | <=.016 | <=.125 | 0.25 | 0.5 | − |
| asaccharolytica | | | | | | | |
| Fusobacterium | 8 | 0.25 | 0.06 | <=.125 | 0.25 | 0.25 | + |
| montiferum | | | | | | | |
| Fusobacterium | 32 | 0.5 | 0.125 | <=.125 | 0.25 | 0.25 | + |
| montiferum | | | | | | | |
| Fusobacterium | 1 | 1 | 0.06 | <=.125 | 0.25 | 0.5 | − |
| montiferum | | | | | | | |
| Fusobacterium | 1 | 1 | 0.06 | <=.125 | 0.5 | 0.5 | − |
| montiferum | | | | | | | |
| Fusobacterium | <=.125 | <=.016 | 0.06 | <=.125 | 0.25 | 0.125 | − |
| nucleatum | | | | | | | |
| Fusobacterium | <=.125 | <=.016 | 0.06 | <=.125 | 0.25 | 0.125 | − |
| nucleatum | | | | | | | |
| Fusobacterium | <=.125 | 0.03 | 0.06 | <=.125 | 0.5 | 0.25 | − |
| nucleatum | | | | | | | |
| Fusobacterium | <=.125 | <=.016 | 0.125 | 0.5 | 0.5 | 0.25 | − |
| nucleatum | | | | | | | |
| Porphyromonas | <=.125 | <=.016 | <=.016 | <=.125 | 0.06 | 0.03 | − |
| gingivalis | | | | | | | |
| Fusobacterium | 1 | 0.5 | 16 | <=.125 | 2 | 2 | − |
| varium | | | | | | | |
| Fusobacterium | 1 | 0.5 | 1 | <=.125 | >16 | >16 | − |
| varium | | | | | | | |
| P acnes | <=.125 | <=.016 | 0.06 | >16 | 0.25 | 0.25 | − |
| P acnes | <=.125 | <=.016 | 0.06 | >16 | 0.25 | 0.25 | − |
| P acnes | <=.125 | <=.016 | 0.06 | >16 | 0.125 | 0.125 | − |
| P acnes | 0.25 | 0.03 | 0.06 | >16 | 0.25 | 0.25 | − |
| Peptostreptococcus | 0.25 | 0.125 | 0.03 | 0.5 | 0.25 | 0.125 | − |
| asaccharolyticus | | | | | | | |
| Fusobacterium | 1 | 1 | 4 | <=.125 | 4 | 4 | − |
| varium | | | | | | | |
| Peptostreptococcus | <=.125 | <=.016 | 0.25 | 2 | 1 | 0.25 | − |
| asaccharolyticus | | | | | | | |
| Peptostreptococcus | 0.25 | 0.06 | 0.125 | 0.5 | 0.125 | 0.06 | − |
| magnum | | | | | | | |
| Peptostreptococcus | <=.125 | <=.016 | 0.06 | 0.25 | 0.125 | 0.06 | − |
| magnum | | | | | | | |
| Peptostreptococcus | 0.25 | 0.06 | 0.125 | 1 | 0.5 | 0.25 | − |
| magnum | | | | | | | |
| Peptostreptococcus | 0.5 | 0.06 | 1 | 0.5 | 0.25 | 0.25 | − |
| magnum | | | | | | | |
| Peptostreptococcus | <=.125 | 0.03 | 4 | 0.25 | 0.5 | 0.25 | − |
| micros | | | | | | | |
| Peptostreptococcus | <=.125 | 0.03 | 0.25 | 0.5 | 4 | 2 | − |
| micros | | | | | | | |
| Peptostreptococcus | <=.125 | 0.03 | 0.125 | 0.5 | 0.5 | 0.5 | − |
| micros | | | | | | | |
| Peptostreptococcus | <=.125 | 0.03 | 0.25 | 0.25 | 16 | 16 | − |
| micros | | | | | | | |
| Peptostreptococcus | <=.125 | 0.03 | 2 | 1 | 1 | 0.5 | − |
| tetradius | | | | | | | |
| Peptostreptococcus | <=.125 | 0.03 | 0.5 | 1 | 0.5 | 0.5 | − |
| tetradius | | | | | | | |
| Peptostreptococcus | <=.125 | <=.016 | 0.25 | 2 | 0.5 | 0.25 | − |
| prevotii | | | | | | | |
| Peptostreptococcus | <=.125 | <=.016 | 0.125 | 1 | 1 | 0.25 | − |
| prevotii | | | | | | | |
| Eubacterium leutum | 1 | 0.25 | 0.06 | 0.25 | 0.25 | 0.5 | − |
| Eubacterium leutum | 1 | 0.5 | 0.25 | 0.25 | 0.5 | 0.5 | − |
| Eubacterium leutum | 1 | 0.5 | 0.25 | 0.5 | 0.5 | 0.5 | − |
| Eubacterium leutum | 1 | 0.5 | 0.06 | 0.5 | 0.5 | 0.5 | − |
| Fusobacterium | 0.5 | 0.25 | 0.03 | 0.25 | 0.5 | 1 | − |
| necrogenes | | | | | | | |

Activity Against Catheter Related Infections

In device-related infections, the correlation between MIC levels and clinical efficacy is poor, leading to the situation that infected implants have to be removed in order to achieve cure. The main characteristics of such infections are the microbial adherence affected by the biofilm and the low growth rate of surface-adherent microorganisms. The discrepancy between the results of routine antibiotic susceptibility testing and treatment success in device-related infections may therefore be due to the fact that bacterial biofilms have different resistance patterns compared with planktonic bacteria. It has been demonstrated that the cure rate in experimental device-related infections can be predicted by the in vitro bactericidal effect of antibiotics on non-growing and adherent bacteria.

The most important anaerobes clinically are the genera of gram negative rods. Bacteroides, especially the B. fragilisgroup is particularly important. The other principal gram negative genera are Prevotella, Fusobacterium, Porphyromonas, Bilophila and Sitterella. Among the gram positive anaerobes, there are cocci (primarily Peptostreptococcus) and spore forming (clostridium) and non spore forming bacilli (Actinomyces and Propionibacteria).

Treatment of anaerobic infections may be difficult. Failure to provide coverage for anaerobes in mixed infections may lead to a poor response or to no response. Many antibacterial agents including aminoglycosides, trimethoprim-sulphamethoxazole, most quinolones and monobactams have poor activity against many or most anaerobes. Four groups of drug are active against majority of anaerobic bacteria of clinical significance: these are nitroimidazole such as metronidazole, carbepenems such as imipenem, chloramphenicol and a combination of 0 lactam and βlactamase inhibitors.

Non spore forming, anaerobic, gram positive bacilli (e.g. Actinomyces, Eubacterium and Propionibacterium) are commonly resistant to metronidazole. Of late, there have been reports of resistance to all the above agents in small number of strains of B. fragilis group. Cefoxitin, clindamycin and braod spectrum penicillins such as ticarcillin or piperacillin also have some anti anaerobic activity. But 15-25% of B. fragilis isolated in the U.S. hospitals are resistant to these drugs. Cefoxitin and clindamycin have relatively weak activity against clostridia other than C. peringens (20-35% of such strains re resistant) and some anaerobic cocci are resistant to clindamycin. Penicillin G is not reliable for treating serious infections involving any of these anaerobic gram negative bacilli because the incidence of β lactamase production among these organisms is high.

To demonstrate the usefulness of novel polymorphic ‘Form A’ in device related infections two tests of experiments have been performed:
1. Inhibition of slime production
2. Activity against glass-adherent bacteria.

To study the effect of polymorphic ‘Form A’ on the inhibition of biofilm production, the following study was carried out as set forth in Blake et al. J. Clinical Microbiol. 2001; 39:544-550; and Polonio et al. Chemother. 2001; 45:3262-3266. Since Mueller Hinton broth does not support the formation of biofilm, trypticase soy broth with 2% glucose was used to stimulate biofilm formation by MRSA 1029/99 and MRSE 879/247 (both recent clinical isolates collected from tertiary care hospital). Bacterial suspensions (in triplicate) were exposed a doubling dilution of antibiotics and incubated overnight at 37° C. with constant shaking (100 rpm). The next day, after aspirating the medium, the biofilm was stained with safranin (0.1%) for 1 hour at room temperature, washed with distilled water, tapped dry and stain-extracted into 200 μl of 0.2M NaoH and the OD measured at 544 nm. The relative inhibition was determined by using the formula:
% inhibition=100-[(OD of treated well/OD of Reference well)×100]

Inhibition of Biofilm formation occurs at a lower concentration for polymorphic ‘Form A’ as depicted in Graphs A to D.

[image]

[image]

Polymorphic ‘Form A’ is active against adherent bacteria:

Linezolid has been shown to be active against nearly all clinically relevant gram positive pathogens, with MIC₉₀ of 2 to 4 μg/ml, while the Cₘₐₓ is 12 to 16 μg/ml. Linezolid is active against all gram positive bacteria, irrespective of their susceptibility to other antibiotics. Though the action is bacteriostatic, it has proven difficult to generate resistant mutants in the laboratory. However, within months of clinical use, resistance in Vancomicin Resistant Enterococci (VRE) and Methicillin Resistant Staphylococcus Aureus (USA) has been reported. The common feature in both reports is the presence of foreign body (catheter) in these patients leading to treatment failure and development of resistant mutants.

We investigated the change in MIC of Linezolid, Vancomycin, Synercid and polymorphic ‘Form A’ in a sintered glass adherent bacteria model with MRSE 879 bacteria and found that though the broth MICs were Linezolid (2 μg/ml), Vancomycin (1 μg/ml), Synercid (0.5 μg/ml) and polymorphic ‘Form A’ (0.5 μg/ml), the concentration which would kill adherent bacteria were Linezolid (32 μg/ml), Vancomycin (8 μg/ml), Synercid (2 μg/ml) and polymorphic ‘Form A’ (2 μg/ml). The change in MIC in broth and on sintered glass adherent bacteria is presented in Graph E.

[image]

Agar Dilution Method for M. tuberculosis:

Antibiotics were incorporated at concentrations of 8, 4, 2, 1, 0.5, 0.25, 0.125, 0.06 and 0.03 μg/ml into plate of Middlebrook 7H10 agar medium supplemented with OADC enrichment (Difco) Test organisms were grown in 7H9 medium (Difco) containing 0.05% Tween 80. After 7 days of incubation at 37° C., the broths were adjusted to 1 MacFarland, the organisms were then diluted 10 fold in sterile water containing 0.05% of Tween 80. The resulting bacterial suspensions were spotted on predried supplemented 7H10 plates. After 21 days of incubation at 37° C., the MICs were recorded as the lowest concentration of the drug that completely inhibited the growth of the organism, and are presented in Tables IV and V.

TABLE IV
MIC (μg/ml)
Mycobacterium tuberculosis

| Drugs | MIC₅₀ | MIC₉₀ | G.M. |
|---|---|---|---|
| Rifampicin | 64 | 64 | 6.35 |
| Isoniazid | 8 | 64 | 3.17 |
| Sparfloxacin | 1 | 2 | 0.53 |
| Clarithromycin | 16 | 32 | 12.69 |
| Linezolid | 8 | 64 | 8 |
| Polymorphic ‘Form A’ | 4 | 64 | 5.44 |

TABLE V
MIC (μg/ml)
Mycobacterium avium intracellulare

| Drugs | MIC₅₀ | MIC₉₀ | G.M. |
|---|---|---|---|
| Rifampicin | 1 | 32 | 1.999 |
| Isoniazid | 32 | 64 | 18.149 |
| Sparfloxacin | 4 | 8 | 3.526 |
| Clarithromycin | 1 | 4 | 1.554 |
| Linezolid | 16 | 64 | 20.587 |
| Polymorphic ‘Form A’ | 8 | 32 | 8.52 |

Examples given below are presented by way of illustration only, and do not limit the scope of the invention.

The free base of Formula I (S)-N-[[3-fluoro-4-[N-1 [4-{2-furyl-(5-nitro)methyl}] piperazinyl]-phenyl]-2-oxo-5-oxazolidinyl]-methyl]acetamide, can be prepared by, for example, following the procedure as described in WO 02/06278.

### EXAMPLE 1

### Preparation of Polymorphic ‘Form A’ of the Compound of Formula I

50 gm of free base of Formula I was dissolved in ethanol (750 ml) by heating at about 60° C. and to this solution was added ethanolic HCl (13.36 ml, 8.9 N) at about 45-50° C. The reaction mixture was cooled to about 10° C., and stirred for about 4 hours. The separated solid was filtered off and dried under vacuum at 60° C. The solid was then digested in ethanol (150 ml) at 70-80° C. for about 4 hours. It was then cooled to about 10° C., the solid was filtered and dried under vacuum at 60-65° C. to give 30 gm of the pure polymorphic ‘Form A’ of compound of Formula I.

### EXAMPLE 2

### Preparation of Polymorphic ‘Form B’ of the Compound of Formula I

7.3 gm of free base of Formula I was dissolved in hot ethanol (130 ml) and cooled to about 20° C. Ethanolic. HCl (2.60 ml, 8.9 N) was added to it. The reaction mixture so obtained was stirred at 20° C. for about 15 minutes. The solid separated was filtered washed with ethanol (30 ml) and dried to give 5.9 gm of pure polymorphic ‘Form B’ of the compound of Formula I.

### EXAMPLE 3

### Preparation of Polymorphic ‘Form A’ of the Compound of Formula I

A solution of free base of Formula I (365 mg, 0.75 mmol, dissolved in 7 ml of ethanol) was heated to about 60-80° C., and then cooled to about 5° C. HCl dissolved in ethanol (0.30 ml, 2.6 N, 0.75 mmol) was added to the reaction mixture at about 5° C. The reaction mixture so obtained was stirred at 5-10° C. for about 2 hours. Solvent was removed completely under vacuum and the residue was digested with dichloromethane, the solid was filtered and crystallized from a mixture of methanol/isopropyl alcohol. The solid obtained was then digested in ethanol (4 ml) at about 80° C. for a time period of about 4 hours. The reaction mixture was cooled to 25-30° C., the solid was filtered and dried under vacuum at about 60° C. to give ‘Form A’ of compound of Formula I.

### EXAMPLE 4

### Preparation of Polymorphic ‘Form A’ of the Compound of Formula I 1.0 gm of (S)-N-[[3-fluoro-4-[N-1 [4-{2-furyl-(5-nitro)methyl]piperazinyl]-phenyl]-2-oxo-5-oxazolidinyl]-methyl]acetamide hydrochloride of Formula I was dissolved in 7 ml of de-mineralized water by heating at 50° C. for few minutes. The solution was cooled to about 40-45° C., and then filtered through 0.2 micron filter paper to remove solid material. Filter paper was washed with water (2.5 ml). To the filtrate was added isopropyl alcohol (40 ml) slowly with stirring at room temperature (25-30° C.). Stirring was continued for about 30 minutes and the solid precipitated was filtered, washed with isopropyl alcohol (5 ml) and then dried under vacuum at about 60° C. for 24 hours to yield 0.85 gm of the pure polymorphic ‘Form A’ of compound of Formula I.

### EXAMPLE 5

### Preparation of Polymorphic ‘Form A’ of the Compound of Formula I

10 gm of (S)-N-[[3-fluoro-4-[N-1 [4-{2-furyl-(5-nitro)methyl})piperazinyl]-phenyl]-2-oxo-5-oxazolidinyl]-methyl]acetamide hydrochloride of Formula I was dissolved in 70 ml of de-mineralized water by heating at about 50° C. for few minutes. The solution was cooled to about 40-45° C., and filtered through 0.2 micron filter paper, and washed with water (10 ml). Ethanol (400 ml) was added slowly to the filtrate at room temperature (25-30° C.). Stirred at room temperature for about 30 minutes, solid separated out. Cooling was continued to about 10-15° C. and kept for 3 hours. The solid was filtered, washed with ethanol (10 ml) and dried under vacuum for 24 hours at about 60° C. to yield 9 gm of the pure polymorphic ‘Form A’ of compound of Formula I.

1. A polymorph ‘Form A’ of(S)-N-[[3-fluoro-4-[N-1-[4-{2-furyl-(5-nitro)methyl)]piperazinyl]-phenyl]-2-oxo-5-oxazolidinyl]-methyl]acetamide hydrochloride having the following 10 most intense X-ray powder diffraction peaks: (2θ):
26.62, 26.20, 24.72, 21.94, 21.18, 20.60, 17.62, 16.84, 16.22, 14.74.

2. The polymorph ‘Form A’ of(S)-N-[[3-fluoro-4-[N-1-[4-{2-furyl-(5-nitro)methyl}]piperazinyl]-phenyl]-2-oxo-5-oxazolidinyl]-methyl]acetamide hydrochloride using an infrared absorption spectrum in potassium bromide with absorption bands expressed in reciprocal centimeters at 3421; 3286; 2967; 1747; 1723; 1668; 1524; 1416; 1354; 1327; 1242; 1170; 1106; 1078; 1022;811 and 749.

3. The polymorph ‘Form A’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride having a differential scanning calorimetry (DSC) endotherm at 211.9° C. (onset at 206.6° C.).

4. A polymorph ‘Form B’ of(S)-N-[[3-fluoro-4-[N-1-[4-{2-furyl-(5-nitro)methyl}]piperazinyl]phenyl}-2-oxo-5-oxazolidinyl]-methyl acetamide hydrochloride having the following X-ray powder diffraction pattern: (2θ):
15.9, 19.1, 20.2, 23.1, 25.7, 26.5, 28.5.

5. The polymorph Form ‘B’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride characterized by an infrared absorption spectrum in potassium bromide having absorption bands expressed in reciprocal centimeters at 3423.2; 2386; 1747; 1654.3; 1519; 1425.9; 1356.2; 1239.2; 1022; 972.1; 811.7 and 750.2.

6. The polymorph Form ‘B’ of(S)-N-[[3-fluoro-4-[N-1-[4-{2-furyl-(5-nitro)methyl}]piperazinyl]-phenyl]-2-oxo-5-oxazolidinyl]-methyl]acetamide hydrochloride having differential scanning calorimetry (DSC) endotherms at 154.9° C. (onset at 148.3° C.) and at 209.2° C. (onset at 207.5° C.).

7. A process for preparing the polymorph ‘Form A’ of (S)-N-[[3-fluoro-4-[N-1-[4-{2-furyl-(5-nitro)methyl}]piperazinyl]-phenyl]-2-oxo-5-oxazolidinyl]-methyl] acetamide hydrochloride, wherein the process comprises:
a) dissolving (S)-N-[[3-fluoro-4-[N-1-[4-{2-furyl-(5-nitro)methyl}]piperazinyl]-phenyl]-2-oxo-5-oxazlidinyl]methyl acetamide in ethanol;
b) adding ethanolic hydrochloric acid;
c) cooling and stirring the reaction mixture;
d) filtering and digesting the solid in ethanol;
e) cooling, filtering and drying the solid to produce polymorph ‘Form A’.

8. The process of claim 7 wherein the cooling of the solid in ethanol after digestion is carried out at a temperature of about 10° C.

9. The process of claim 7 wherein the drying of the product is carried out under vacuum at a temperature ranging from about 60-65° C.

10. A process for preparing the polymorph ‘Form B’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride, wherein the process comprises:
a) dissolving (S)-N-[[3-fluoro-4-[N-1-[4-{2-furyl-(5-nitro)methyl}]piperazinyl]-phenyl]-2-oxo-5-oxazolidenyl]methyl acetamide in ethanol;
b) cooling the solution and adding ethanolic hydrochloric acid;
c) stirring the reaction mixture;
d) filtering the solid to produce polymorph ‘Form B’.

11. The process of claim 10 wherein the cooling is carried out at a temperature of about 20° C.

12. A process for preparing the polymorph ‘Form A’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride, wherein the process comprises:
a) dissolving (S)-N-[[3-fluoro-4-[N-1-[4-{2-furyl-(5-nitro)methyl}]piperazinyl]-phenyl]-2-oxo-5-oxazolidinyl]methyl acetamide in ethanol;
b) adding a mixture of hydrochloric acid in ethanol;
c) removing the solvent and digesting the residue in dichloromethane;
d) filtering and crystallizing the solid;
e) digesting the solid in ethanol;
f) cooling, filtering and drying the solid to produce polymorph ‘Form A’.

13. The process of claim 12 wherein the crystallization of the solid is carried out in a solvent selected from the group comprising of methanol and isopropyl alcohol.

14. The process of claim 12 wherein the cooling is carried out at a temperature of about 25-30° C.

15. The process of claim 12 wherein the drying of solid is carried out under vacuum at a temperature ranging from about 60-65° C.

16. A process for preparing the polymorph ‘Form A’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride, wherein the process comprises:
a) dissolving (S)-N-[[3-fluoro-4-[N-1-[4-{2-furyl-(5-nitro)-methyl}]piperazinyl]-phenyl]-2-oxo-5-oxazolidinyl]methyl acetamide hydrochloride in de-mineralized water;
b) adding isopropyl alcohol;
c) stirring and filtering the solid;
d) drying the solid to produce polymorph ‘Form A’.

17. The process of claim 16 wherein the drying of solid is carried out under vacuum at a temperature of about 60° C.

18. A process for preparing the polymorph ‘Form A’ of (S)-N-[[3-fluoro-4-[N-1-[4-{2-furyl-(5-nitro)methyl}]piperazinyl]-phenyl]-2-oxo-5-oxazolidinyl]-methyl]acetamide hydrochloride, wherein the process comprises:
a) dissolving (S)-N-[[3-fluoro-4-[N-1-[4-{2-furyl-(5-nitro)-methyl}]piperazinyl]-phenyl]-2-oxo-5-oxazolidinyl]methyl acetamide hydrochloride in de-mineralized water;
b) adding ethanol;
c) stirring, cooling and filtering the solid;
d) drying the solid to produce polymorph ‘Form A’.

19. The process of claim 18 wherein the drying of solid is carried out under vacuum at a temperature of about 60° C.

20. A pharmaceutical composition comprising a compound according to any one of the claims 1, 2, 3, 4, 5 or 6 and a pharmaceutically acceptable carrier.

21. A method of treating or preventing microbial infections in a mammal comprising administering to the said mammal, a compound according to any one of the claims 1, 2, 3, 4, 5 or 6.

22. The method of treating or preventing microbial infections in a mammal comprising administering to the said mammal, a pharmaceutical composition according to claim 20.

23. A method of treating or preventing aerobic and anaerobic bacterial infections in a mammal comprising administering to the said mammal, a therapeutically effective amount of a compound according to any one of the claims 1, 2, 3, 4, 5 or 6.

24. The method of treating or preventing aerobic and anaerobic bacterial infections in a mammal comprising administering to the said mammal, a therapeutically effective amount of a pharmaceutical composition according to claim 20.

25. A method of treating or preventing catheter infections and foreign body or prosthesis infections in a mammal comprising administering to the said mammal, a therapeutically effective amount of a compound according to any one of the claims 1, 2, 3, 4, 5 or 6.

26. The method of treating or preventing catheter infections and foreign body or prosthesis infections in a mammal comprising administering to the said mammal, a therapeutically effective amount of a pharmaceutical composition according to claim 20.

27. The method according to claim 21 or 22 wherein the microbial infections are caused by gram positive and gram negative bacteria.

28. The polymorph ‘Form A’ of claim 1 having the following 20 most intense X-ray powder diffraction peaks: (2θ):
31.48, 28.60, 28.14, 26.62, 26.20, 24.72, 23.52, 22.84, 22.48, 21.94, 21.18, 20.60, 20.00, 19.74, 17.62, 16.22, 16.84, 14.74, 13.20, 12.86.
